(19) United States
(12) Patent Application Publication
(10) Pub. No.: US 2026/0308176 A1
(43) Pub. Date: Oct. 8, 2026

(54) VOLTAGE SENSITIVE THIAZOLOTHIAZOLE DYES AND ASSOCIATED SYNTHETIC METHODS

(71) Applicant: The University of North Carolina at Charlotte, Charlotte, NC (US)

(72) Inventors: Michael G. Walter, Charlotte, NC (US); Pranamita Chakraborti, Charlotte, NC (US)

(21) Appl. No.: 19/255,533

(22) Filed: Jun. 30, 2025

Related U.S. Application Data
(60) Provisional application No. 63/665,817, filed on Jun. 28, 2024.

(51) Int. Cl. C07D 513/04; A61K 49/00

(52) U.S. Cl. CPC C07D 513/04; A61K 49/0021

(57) ABSTRACT

In one aspect, π-conjugated asymmetric molecular dyes are described herein comprising a donor (D)-acceptor (A) architecture across a thiazolothiazole bridge. In another aspect, methods of making thiazolothiazole-based compounds are described herein which obviate the need for chromatographic isolation of product.

### RELATED APPLICATION DATA

The present application claims priority pursuant to 35 U.S.C. § 119(e) to U.S. Provisional Patent Application Ser. No. 63/665,817 filed Jun. 28, 2024 which is incorporated herein by reference in its entirety.

### STATEMENT REGARDING FEDERALLY SPONSORED RESEARCH

This invention was made with government support under Grant No. 1R15GM140392-01 and Grant No. 2R15GM140392-02 awarded by the National Institutes of Health. The government has certain rights in the invention.

### FIELD

The present invention relates to voltage sensitive dyes and, in particular, to voltage sensitive asymmetric thiazolothiazole-based compounds and associated synthetic methods.

### BACKGROUND

All biological membranes harbor a transmembrane potential, or voltage, that is pivotal to their function. This fundamental feature arises because the biological fluids they separate are highly asymmetric in terms of the concentrations of the major physiological ions e.g., Na⁺, K⁺, Ca²⁺, and Cl⁻. This sets up an electrochemical gradient across the membrane and the opening of membrane-resident ion channels leads to the flow of specific ions, thus giving rise to membrane potential. While membrane potential may be measured using electrophysiology, the method is limited by its invasiveness and lack of spatial information on membrane potential. Genetically encoded voltage indicators (GEVIs) provide spatial resolution, but are constrained by their lower brightness and fast photobleaching that limits long-term monitoring of membrane-potential dynamics. Hence there has been a renewed focus on developing VSDs that can fluorescently report on membrane potential by diverse mechanisms.

### SUMMARY

In view of the foregoing, π-conjugated asymmetric molecular dyes are described herein comprising a donor (D)-acceptor (A) architecture across a thiazolothiazole bridge. In some embodiments, an asymmetric thiazolothiazole-based compound is of Formula I:

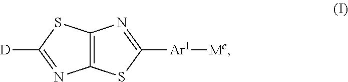

wherein D is an electron donor, Ar¹ is selected from the group consisting of arylene and heteroarylene, and M^{c} is a charged moiety. In some embodiments, M^{c} is cationic. For example, M^{c} can comprise a quaternary ammonium group. Moreover, in some embodiments, M^{c} is of the formula:

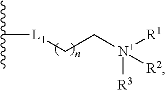

wherein L₁ is selected from the group consisting of a direct bond, —C(O)O—, —C(O)—, —R⁴—C(O)O—, and —R⁵—C(O)—, and wherein R¹-R³ are independently selected from the group consisting of hydrogen, alkyl, cycloalkyl, and alkenyl, and wherein R⁴ and R⁵ are independently selected from the group consisting of alkyl and alkenyl, and wherein n is an integer from 0 to 10.

Alternatively, M^{c} can be anionic. When anionic, M^{c} can comprise an anionic acid group, including carboxylate or sulfonate. In some embodiments, M^{c} is of the formula:

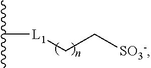

wherein L₁ is selected from the group consisting of a direct bond, —C(O)O—, —C(O)—, —R¹—C(O)O—, and —R²—C(O)—, and wherein R¹ and R² are independently selected from the group consisting of alkyl and alkenyl, and wherein n is an integer from 0 to 10. As described further herein, the anionic or cationic character of M^{c} can facilitate anchoring of the thiazolothiazole-based compounds to biomolecular structures, including phospholipid bilayers of cells, and/or liposomes. When cationic, M^{c} can interact with anionic phosphate groups of membrane lipids. When anionic, M^{c} can interact with quaternary ammonium groups of the membrane, such as those associated with phosphatidyl choline and sphingomyelin. In some embodiments, ionic interactions between thiazolothiazole-based compounds described herein and charged moieties of the cell membrane can preclude passage of the thiazolothiazole-based compounds into the cell.

Additionally, D, in some embodiments, is hydrophobic. The hydrophobic nature of D can facilitate insertion of D into the hydrophobic interior of the cell membrane or other phospholipid bilayers. In some embodiments, D is selected from the group consisting of aryl and heteroaryl. If desired, the aryl and heteroaryl comprise one or more substituents further increasing the hydrophobicity of D. In some embodiments, the substituents are selected from the group consisting of alkylamine and arylamine. In some embodiments, D is of the formula:

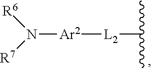

wherein L₂ is selected from the group consisting of a direct bond, alkylene, cycloalkylene, and alkenylene, and wherein Ar² is selected from the group consisting arylene and heteroarylene, and wherein R⁶ and R⁷ are independently selected from the group consisting of hydrogen, alkyl, cycloalkyl, alkenyl, alcohol, aryl, and heteroaryl. In some embodiments, for example, D is of the formula:

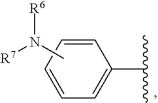

wherein R⁶ and R⁷ are independently selected from the group consisting of hydrogen, alkyl, cycloalkyl, alkenyl, alcohol, aryl, and heteroaryl.

In another aspect, biological compositions are described herein. A biological composition, in some embodiments, comprises cells or cellular components, and a marker associated with membranes of the cells or cellular components, the marker comprising a thiazolothiazole-based compound of Formula I:

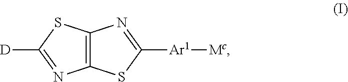

wherein D is an electron donor, Ar¹ is selected from the group consisting of arylene and heteroarylene, and M^{c} is a charged moiety. The thiazolothiazole-based compounds of Formula I can interact with cell membranes as described above and can have any structure described herein. In some embodiments, fluorescence of the thiazolothiazole-based compound increases or decreases relative to voltage changes in a local environment of the thiazolothiazole-based compound. Fluorescent intensity, for example, can vary in response to changes in potential of the cell membrane. In this way, fluorescent intensity of the thiazolothiazole-based compounds can be correlated with membrane potential.

In another aspect, methods of making thiazolothiazole-based compounds are described herein. In some embodiments, a method comprises reacting dithiooxamide with a first aryl aldehyde and a second aryl aldehyde in a polar protic organic solvent to provide the thiazolothiazole-based compound. The thiazolothiazole-based compound is subsequently isolated without chromatographic separation. Accordingly, the thiazolothiazole-based compound can be isolated from other species in the reaction mixture, including unreacted reagents, without the need for chromatographic separation. Obviating the requirement of product isolation and/or purification by chromatographic separation techniques greatly simplifies compound synthesis and collection. In embodiments of methods described herein, the first aryl aldehyde and/or the second aryl aldehyde can be replaced with a heteroaryl aldehyde, as desired.

These and other embodiments are further described in the following detailed description.

### BRIEF DESCRIPTION OF THE DRAWINGS

FIG. 1A illustrates mixed substituent dibutylaminophenyl-carbomethoxyphenyl asymmetric thiazolo[5,4-d]thiazole (asym-TTz) derivative (Bu₂N-TTz-CO₂CH₃-Compound 1) accessed exclusively using n-propanol and long reaction times.

FIG. 1B provides Sequential synthesis of Bu₂N-TTz-CO₂(NPr) (Compound 3) starting from Compound 1.

FIG. 2A illustrates solvatofluorochromism of Compound 1 in non-polar solvents (hexane) to polar solvents (MeOH). a=Hexane; b=Toluene; c=CHCl₃; d=CH₂Cl₂; e=THF; f=EtOAc; g-Acetone; h=MeCN; i=DMSO; j-MeOH.

FIG. 2B illustrates DFT calculations and energies (eV) for highest occupied (HOMO) and lowest unoccupied (LUMO) molecular orbitals of Compound 3.

FIG. 2C provides fluorescence spectra of Compound 2 in various solvents upon excitation at 415 nm.

FIG. 2D s a Lippert-Mataga plot from various solvents showing the long (LWB) and short (SWB) wavelength band transition dipole moments (Au) in the fluorescent spectra as a function of solvent polarity.

FIG. 2E is time-correlated single photon counting data (TCSPC)-fluorescence lifetimes of Compound 3 at 405 nm in various solvents.

FIG. 3A quantifies cytotoxicity of Compound 3 given by an MTT assay.

FIG. 3B illustrates Fluorescence images of HeLa cells co-labeled with CellMask™ and Compound 3.

FIG. 3C are pixelwise single cell maps of the ratio of fluorescence intensities taken at the indicated potential (Fₓ) and that at −100 mV (F₋₁₀₀)

FIG. 3D illustrates fractional change in fluorescence (F/Fₘₐₓ) versus membrane potential applied via whole cell patch clamping. n=5 and 10 for resting cells and voltage clamped cells.

FIG. 3E illustrates images of Compound 3 labeled HeLa cells at the indicated times showing stability of cell membrane localization.

FIG. 4A illustrates normalized emission spectra of Compound 3 labeled HeLa cells excited at 440 nm at resting potential and at +100 mV.

FIG. 4B provides computed conformation of Compound 3 in the ground state and in the excited state under an applied potential of +100 mV. ω is the twist between the S—C—C—C (red) or N—C—C—C(green) bonds.

FIG. 5 illustrates various thiazolothiazole-based compounds described herein.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

“Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). In some embodiments the alkyl can be a lower alkyl. “Lower alkyl” refers to a straight or branched chain alkyl having from 1 to 3, or from 1 to 5, or from 1 to 8 carbon atoms. Representative examples of alkyl include, but are not limited to, methyl, ethyl, npropyl, iso-propyl, n-butyl, sec-butyl, iso-butyl, tert-butyl, n-pentyl, isopentyl, neopentyl, nhexyl, 3-methylhexyl, 2,2-dimethylpentyl, 2,3-dimethylpentyl, n-heptyl, n-octyl, n-nonyl, ndecyl, and the like. In some embodiments, alkyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy. Representative examples of halo substituted alkyls include, but are not limited to, fluoromethyl, difluoromethyl and trifluoromethyl.

“Alkenyl,” as used herein, refers to a straight or branched chain hydrocarbon containing from 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. In some embodiments, alkenyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Cycloalkyl,” as used herein, refers to a saturated cyclic hydrocarbon group containing from 3 to 8 carbons or more. Representative examples of cycloalkyl include, cyclopropyl, cyclobutyl, cyclopentyl, cyclohexyl, cycloheptyl, and cyclooctyl. In some embodiments, cycloalkyl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy. A representative example of a substituted cycloalkyl include epoxide.

“Heterocycle,” as used herein, refers to a monocyclic, bicyclic, or tricyclic ring system comprising at least one heteroatom. Monocyclic heterocycle ring systems are exemplified by any 4-, 5-, 6- or 7-member ring containing 1, 2, 3, or 4 heteroatoms independently selected from the group consisting of: O, N, and S. The 4-member ring has 0 to 1 double bond, the 5-member ring has from 0 to 2 double bonds, and the 6 and 7 member rings have from 0 to 3 double bonds.

“Aryl” as used herein refers to a ring system having one or more aromatic rings. A multicyclic ring system includes fused rings and unfused rings or various combinations thereof. Representative examples of aryl include azulenyl, indanyl, indenyl, naphthyl, phenyl, tetrahydronaphthyl, and the like. The aryl groups of this invention can be substituted with 1, 2, 3, 4, or 5 substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfamyl, sulfo, sulfonate, —NR′R″ (wherein, R′ and R″ are independently selected from hydrogen, alkyl, alkylcarbonyl, aryl, arylalkyl and formyl), and —C(O) NR′R″ (wherein R′ and R″ are independently selected from hydrogen, alkyl, alkylcarbonyl, aryl, arylalkyl, and formyl). In some embodiments, aryl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Heteroaryl” as used herein refers to an aromatic monocyclic or multicyclic ring system in which one or more of the ring atoms is an element other than carbon, such as nitrogen, boron, oxygen and/or sulfur. The multicyclic ring system includes fused rings and unfused rings or various combinations thereof. The multicyclic ring system can also include one or more aryl moieties in conjunction with one or more heteroaryl moieties. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, benzofuranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 4 suitable substituents, as chemically feasible. For example, the heteroatom S may be substituted with one or two oxo groups, which may be shown as ═O. In some embodiments, heteroaryl groups as described herein are optionally substituted (e.g., from 1 to 3 or 4 times) with independently selected halo, hydroxy, acyl, alkyl, alkenyl, alkynyl, cycloalkyl, heterocycle, aryl, heteroaryl, alkoxy, amino, amide, thiol, sulfide, sulfone, sulfoxide, carbonyl, or carboxy.

“Halo” as used herein, alone or in combination, refers to elements of Group VIIA or Group 17 of the Periodic Table (halogens). Depending on chemical environment, halo can be in a neutral or anionic state.

Terms not specifically defined herein are given their normal meaning in the art.

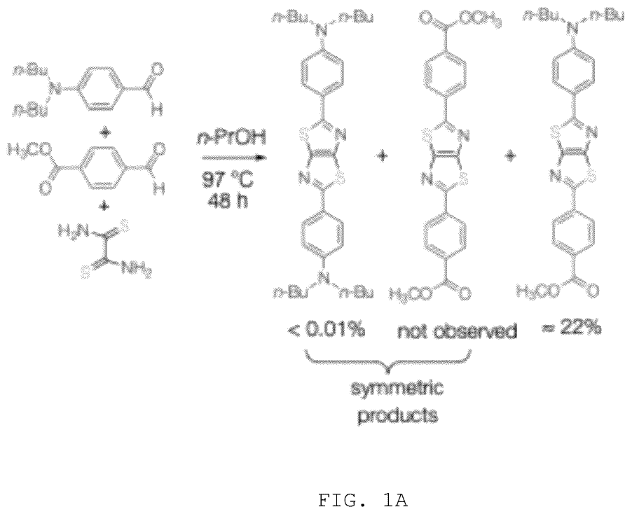

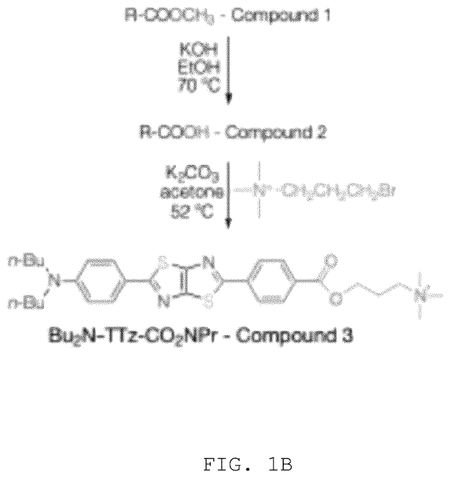

Thiazolothiazole-based compounds are described herein which, in some embodiments, exhibit advantageous molecular and electronic structures for characterizing cell membrane properties, including membrane potentials. In some embodiments, the thiazolothiazole-based compounds are of Formula I:

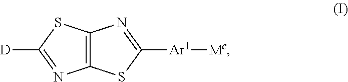

wherein D is an electron donor, Ar¹ is selected from the group consisting of arylene and heteroarylene, and M^{c} is a charged moiety. As evidenced by the structure of Formula I, the thiazolothiazole-based compounds are asymmetric. FIG. 1 illustrates various asymmetric thiazolothiazole-based compounds described herein. For the thiazolothiazole-based compounds of FIG. 1, each R group is independently selected from the moieties in the table of FIG. 1. Accordingly, the R groups of the thiazolothiazole-based compounds of FIG. 1 can be the same or different.

Thiazolothiazole-based compounds described herein, in some embodiments, can exhibit a difference between the highest occupied molecular orbital (HOMO) and lowest unoccupied molecular orbital (LUMO) of at least 1 eV. In some embodiments, the HOMO/LUMO offset is 1 eV to 4 eV or 1 eV to 3 eV.

Thiazolothiazole-based compounds described herein, in some embodiments, can display a change in dipole moment between ground and excited states of at least 15 D. The change in dipole moment can also have a value selected from Table I.

TABLE I
Change in Dipole Moment (D)
15-30
15-40
16-40
17-40
18-40
20-40
20-30

Thiazolothiazole-based compounds described herein are also fluorophores, in some embodiments. Thiazolothiazole-based compounds, for example, can have a peak absorption in the range of 400 nm to 650 nm, and a peak emission in the range of 400 nm to 800 nm. In some embodiments, the absorption profile does not overlap or substantially overlap with the emission profile. Absorption and emission profiles do not substantially overlap if less than 10 percent or less than 5 percent of the profiles have overlap. Additionally, thiazolothiazole-based compounds described herein can exhibit solvent dependent fluorescence lifetimes. Fluorescence lifetime can vary relative to solvent polarity, in some embodiments. The thiazolothiazole-based compounds can display solvatofluorochromism, exhibiting a Stokes shift of 0.25 to 0.75 eV, in some embodiments. In some embodiments, the Stokes shift is at least 0.3 eV. The thiazolothiazole-based compounds can also exhibit quantum yields greater than 80 percent, greater than 85 percent, or greater than 90 percent in non-polar solvents.

The thiazolothiazole-based compounds of Formula I can interact with lipid species of the cell membrane as described above. In some embodiments, fluorescence of the thiazolothiazole-based compound increases or decreases relative to voltage changes in a local environment of the thiazolothiazole-based compound. Fluorescent intensity, for example, can vary in response to changes in potential of the cell membrane. In this way, biological compositions can employ fluorescent intensity of the thiazolothiazole-based compounds to characterize membrane potential.

In another aspect, methods of making symmetric and asymmetric thiazolothiazole-based compounds are described herein. In some embodiments, a method comprises reacting dithiooxamide with a first aryl aldehyde and a second aryl aldehyde in a polar protic organic solvent to provide the thiazolothiazole-based compound. The thiazolothiazole-based compound is subsequently isolated without chromatographic separation and/or any other chemical purification steps. Any polar protic organic solvent enabling chromatographic-free isolation of the thiazolothiazole-based compound can be employed. In some embodiments, the polar protic organic solvent has a boiling point less than 100° C. The polar protic organic solvent, for example, can be a low molecular weight and/or short chain alcohol, such as propanol, including 1-propanol. In some embodiments, suitable alcohols have a boiling point above 60° C. Additional polar protic organic solvents can including n-propanol, isopropyl alcohol, n-butanol, t-butanol, ethanol, pentanol, and various mixtures thereof.

In some embodiments of methods described herein, solvent of the reaction mixture comprises one or more aprotic solvents. Suitable aprotic solvents can have a boiling point in the range of 60° C. to 115° C., in some embodiments. An aprotic solvent can comprise benzene, toluene, 1,4-dixoane, chloroform, ethyl acetate, tetrahydrofuran (THF), acetonitrile and various mixtures thereof.

Additionally, the first aryl aldehyde and a second aryl aldehyde may have the same structure or differing structures to provide symmetric or asymmetric thiazolothiazole-based compounds, respectively. Moreover, the first and/or second aryl aldehyde can be provided in molar excess relative to the dithiooxamide, in some embodiments.

In embodiments of methods described herein, the first aryl aldehyde and/or the second aryl aldehyde can be replaced with a heteroaryl aldehyde. In some embodiments, wherein the reaction mixture comprises a first heteroaryl aldehyde and second heteroaryl aldehyde, the first and second heteroaryl aldehydes can be the same or different.

In some embodiments, methods described herein further comprise functionalizing the thiazolothiazole-based compound with a cationic or anionic moiety. Identity of the cationic or anionic moiety can be M^{c} as provided above.

These and other embodiments are further illustrated in the following non-limiting examples.

### Example 1—Asymmetric Thiazolothiazole-based Compounds

As described in the methods herein, asym-TTz synthesis has been improved by exploring various conditions and solvent systems for the bicyclization reaction of dithiooxamide with aromatic aldehydes (FIG. 1). The synthesis is usually carried out in dimethylformamide (DMF) at high temperatures (~150° C.) which causes product precipitation with yields between 40-50%. In addition, the use of high boiling solvents like DMF complicates purification. 1-propanol was, therefore, examined as a solvent. Using 1-propanol for the reaction allows for lower temperatures (97° C.) and easier solvent removal. A molar excess of aldehydes relative to dithiooxamide (donor-aldehyde:dithiooxamide:acceptor-aldehyde=1.6:1.45:1.6) was used.

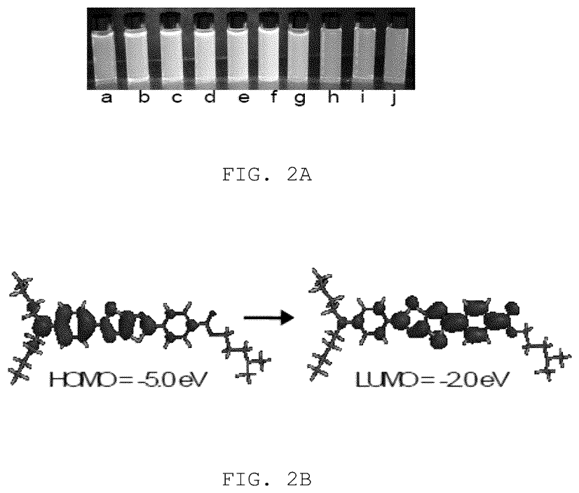

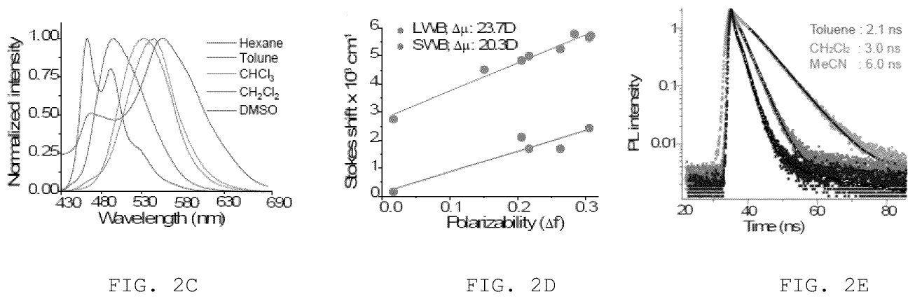

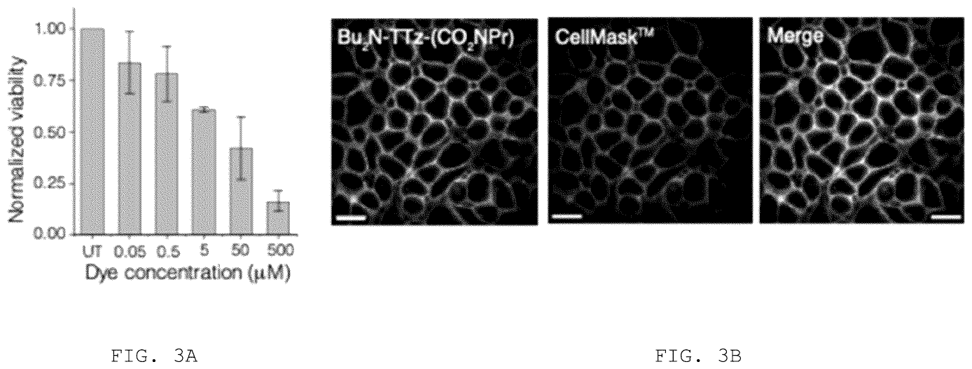

Products precipitated readily out of the reaction mixture, were collected, filtered, and rinsed with cold 1-propanol. While alcohol solvents and lower temperatures are uncommon in TTz synthesis, short-chain alcohols facilitate synthesis scale-up. Interestingly, of the three possible products from the reaction (FIG. 1), the symmetric dicarbomethoxyphenyl TTz and the symmetric dibutylaminophenyl TTz were either undetected or formed in trace amounts (<0.01%). Though the overall yields were moderately low for the asymmetric (Bu₂N-TTz-CO₂CH₃-Compound 1) derivative (~22%), the immediate isolation of the pure asymmetric derivative is hugely advantageous compared to the mixed substituent reaction used to access the first generation of asym-TTz VSDs, since no purification step is involved here and the crude material from the reaction, once filtered, is all product. No such asymmetric TTz reaction has been reported in literature where the direct isolation of any target asymmetric product from a one-step mixed-pot reaction has been made possible. In general, the reaction conditions were different from those described for earlier asymmetric TTz dyes, features the use of 1-propanol, lower reaction temperature, and importantly, no chromatographic separations. The present asym-TTz dyes exhibit strong solvatofluorochromism with fluorescence Stokes shifts ranging from 0.24 eV to 0.71 eV in a range of solvents (FIG. 2A)

Compound 1 could be used to easily form Bu₂N-TTz-CO₂H (Compound 2) by ester to acid conversion. Compound 2, subsequently, could then be used to form Bu₂N-TTz-CO₂(NPr) (Compound 3), as illustrated in FIG. 1B. The latter was designed for cell membrane localization and voltage sensing studies, where the positively charged trimethylammonium group was incorporated to orient the dye in the cell membrane and the two n-butyl groups facilitated membrane anchoring. Overall, this series of derivatives and related compounds can be readily accessed in significant quantities without the need for chromatographic purification.

### Example 2—Asymmetric Thiazolothiazole-based Compound Characterization

Investigations of the photophysical properties of asym-TTz derivatives included UV-vis absorption, steady-state and time-resolved fluorescence studies in various organic solvents (Table 2). Asym-TTz dyes showed solvatofluorochromic emission characteristics (FIG. 2A) indicative of strong, push-pull intramolecular charge transfer (ICT) properties. The small shifts in absorption peaks (λ_{abs}) in their spectra indicate negligible solvent effects on the ground state dipole moment. All asym-TTz derivatives showed significant Stokes shifts between 0.24 eV and 0.71 eV with increasing polarity of the organic solvent, with slightly more red-shifted absorbance and emission values for Compound 3.

The emission spectrum for Compound 1 and Compound 2 in hexane show clear vibrational energy levels around 460 nm, 490 nm, and a broader shoulder at 530 nm, consistent with other asym-TTzs. These peaks become less resolved and eventually merge as solvent polarity increases. Importantly, this class of push-pull asym-TTzs shows the presence of a multiple band, dual fluorescent twisted intramolecular charge transfer (TICT) and a locally excited (LE) state in more polar solvents such as DMSO (FIG. 2C). We recently were able to associate the dual fluorescence of asym-TTzs to a short wavelength band (SWB) that is associated with emission from a twisted excited state, where the dialkyl/diphenylamino substitutent is twisted relative to the plane of the TTz phenyl ring, and the longer wavelength band (LWB) is associated with emission from a planar excited state. Density functional theory (DFT) modeling of the absorbance features provided strong evidence for the TTz dyes existing in a twisted ground state. Their fluorescence lifetimes (TCSPC) depended on the polarity and the protic nature of the solvents. Increasingly polar solvents lowered the energy of the excited state with some indications of hydrogen bonding effects given the decrease in the fluorescence lifetime in protic solvents. The longest fluorescence lifetime observed was 6.0 ns in the case of the Compound 3 derivative in acetonitrile (FIG. 2E), and for all three derivatives, radiative rates decreased with increasing polarity, while the non-radiative rates followed the reverse trend.

The quantum yields were very high in non-polar solvents (e.g. Φ_{Hex}=0.98 in Compound 1) and low in polar solvents (e.g. Φ_{MeOH}=0.04 in Compound 1). It was estimated that the unexpectedly low quantum yields of Compound 2 and Compound 3 in certain non-polar solvents (e.g. hexane) is due to their low solubility. Compound 3, in particular, due to the presence of the positive charge, shows extremely low solubility in many of the organic solvents examined. Therefore, its solvatofluorochromic behavior is lessened in comparison to Compound 1 or Compound 2, despite the push-pull chemical nature. However, this is not expected to affect the voltage sensitivity of this dye. This is due to the fact that Compound 3, regardless of its poor solubility in most organic solvents, positions itself very well by anchoring strongly onto the cellular membrane, which is key to the voltage sensing phenomenon. Since cellular membranes have a hydrophobic center where VSDs localize, high quantum yields in non-polar environments are desirable as it implies high fluorescence in contexts where VSDs are expected to function.

TABLE 2
Optical Properties of asym-TTz Dye (Compound 3) in Various Solvents

| | | | | | | Stokes | Quantum | | | Non- |
|---|---|---|---|---|---|---|---|---|---|---|
| Compound | | Abs. | Abs. | Em. | Em. | shift | yield | Fluorescence | Radiative | radiative |
| 3 | Solvent | (nm) | (eV) | (nm) | (eV) | (eV) | (Φ_{F}) | lifetime (ns) | rate (s⁻¹) | rates (s⁻¹) |
| Bu₂N-TTz- | Toluene | 445 | 2.79 | 507 | 2.45 | 0.34 | 0.08 | 2.1 | 0.4 × 10⁸ | 4.5 × 10⁸ |
| CO₂(NPr) | | | | | | | | | | |
| | DCM | 436 | 2.85 | 557 | 2.23 | 0.62 | 0.32 | 3.0 | 1.1 × 10⁸ | 2.3 × 10⁸ |
| | MeCN | 429 | 2.90 | 566 | 2.20 | 0.70 | 0.11 | 6.0 | 0.2 × 10⁸ | 1.5 × 10⁸ |

To quantify the intramolecular charge transfer (ICT) in these new asym-TTz dyes, the Lippert-Mataga equation was used (Eq. 1):

$${v}_{a} - {v}_{f} = \frac{{( {μ}^{*} - μ )}^{2}}{2 {πϵ}_{0} {hca}^{3}} Δ f + const . ; Δ f = ( \frac{ϵ - 1}{2 ε + 1} - \frac{{η}^{2} - 1}{2 {η}^{2} + 1} )$$

where vₐ and v_{f}=wavenumbers of the absorption and emission peaks in cm⁻¹, respectively; μ* and μ=excited state and ground state dipoles, respectively; ε₀=vacuum permittivity; h=Planck's constant; c=speed of light; a=Onsager cavity radius; Δf=orientation polarizability; ε=relative permittivity; and η=refractive index. All calculations for dipoles and Onsager cavity radii were performed using Spartan 16 software. The Lippert-Mataga plots showed correlation values >0.9, indicating negligible solute-solvent interactions, or solute polarizability. Some of the highest ever observed change in dipole moments were obtained in these new push-pull asym-TTz dyes. Therefore it was expected that these dyes show high environmental sensitivity, predisposing them to cellular sensing applications. Transition dipole moments were quantified separately for both the SWB and the LWB of Compound 3, which were clearly detectable in certain solvents. The strong electron withdrawing nature of the trimethylammonium group is likely responsible for these differences in the LWB and SWB photophysical characteristics. The change in dipole moment (23.7 D) of LWB exceeded that of the SWB (20.3D), and a closer Lippert-Mataga fit (FIG. 2D) was obtained for the LWB. The LWB is more influenced by the polarizability of the solute and the solute-solvent interactions than the LWB, consistent with other asym-TTz derivatives.

TABLE 3
Ground and Excited State Dipole Moments

| | | Ground | Excited | |
|---|---|---|---|---|
| | Onsager | State | State | |
| | Radius | Dipole | Dipole | Change in |
| | a^{a} | Moment | Moment | Dipole |
| Compounds (1-3) | (Aº) | (μ)^{a} (D) | (μ*)^{b} (D) | (Δμ)^{b} (D) |
| Bu₂N-TTz-CO₂CH₃ | 8.28 | 6.82 | 31.9 | 25.1 |
| Bu₂N-TTz-CO₂H | 8.28 | 7.52 | 30.9 | 23.3 |
| Bu₂N-TTz-CO₂(NPr) | 8.28 | 6.45 | 30.2 | 23.7 |
| (LWB) | | | | |
| Bu₂N-TTz-CO₂(NPr) | 8.28 | 6.45 | 26.8 | 20.3 |
| (SWB) | | | | |

^{a}Calculated in Spartan 2016 software. ^{b}Semi-empirically calculated using Lippert-Mataga equation (Eq. 1).

DFT calculations were carried out in Spartan 16 using B3LYP/6-31G* for the esters and the carboxylic acid derivative to determine highest occupied molecular orbital (HOMO) and lowest occupied molecular orbital (LUMO) energies. These revealed that the dibutylaminophenyl/carboxyphenyl substituents provide ground and excited state dipoles with a large electron density shift from the HOMO to the LUMO (FIG. 2B). This is consistent with UV-vis and fluorescence measurements showing an absorbance onset that is red-shifted by ~20 nm compared to the first generation asym-TTz dyes. DFT calculations show the HOMO residing over the dibutylaminophenyl (Bu₂N) donor group whereas the LUMO resides on carboalkoxyphenyl acceptor (FIG. 2B). The donor-acceptor nature of the dye and spatially separated HOMO/LUMO levels show good electronic overlap across the planar TTz core.

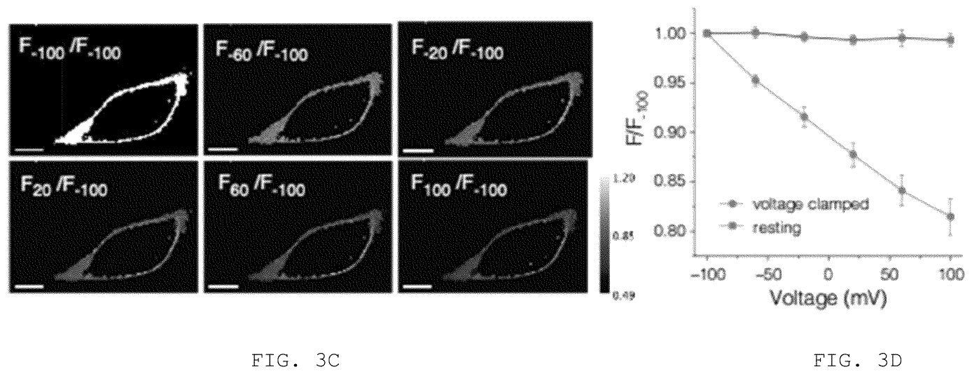

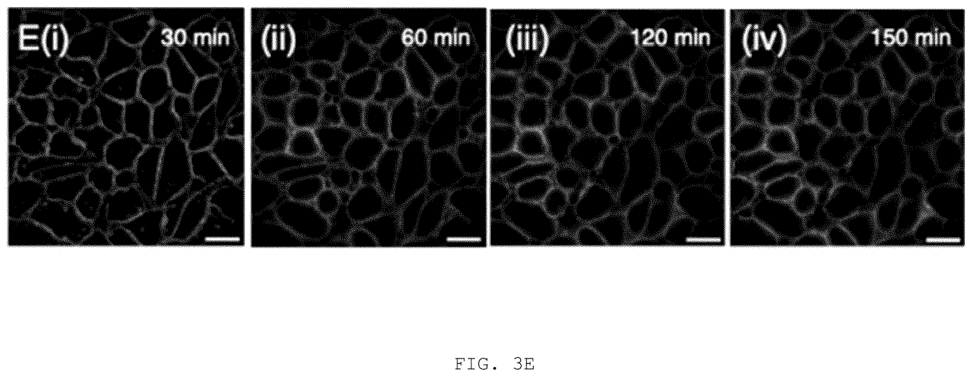

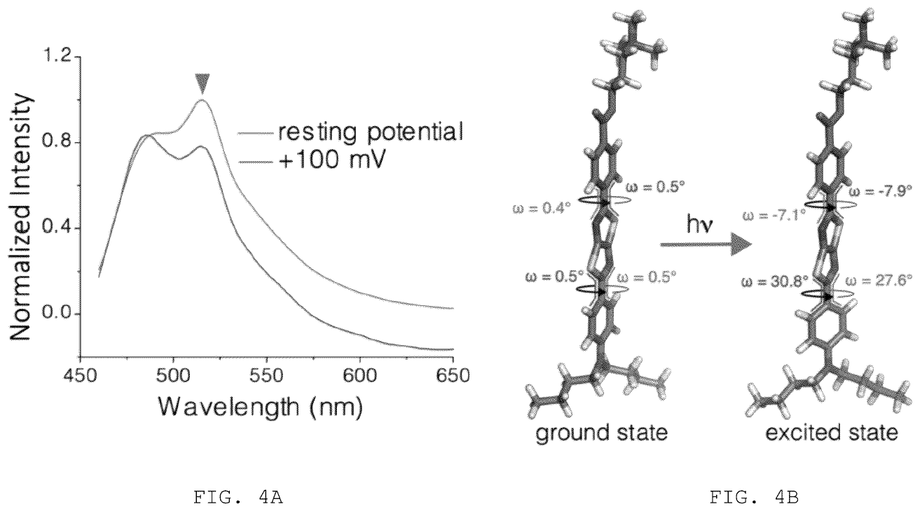

The ability of Compound 3 was evaluated to report on membrane potential in live cells. Compound 3 showed negligible cell toxicity≤500 nM, which is the concentration used for all future studies (FIG. 3A). HeLa cells were co-labeled with an admixture of the plasma membrane marker, CellMask™ and 500 nM Compound 3 (in 1% DMSO/water), and both dyes were imaged by fluorescence microscopy. Compound 3 and CellMask™ showed excellent colocalization in the plasma membrane (FIG. 3B). The Pearson correlation coefficient was as high as 0.99 for Compound 3 and an average of 0.97 was obtained using 15 cells from FIG. 3B (using Fiji—Image J). It was expected that the hydrophobic dibutylaminophenyl donor side is embedded in the outer leaflet of the cell membrane while the hydrophilic trimethylammonium charged group is oriented towards the membrane/water interface at the cell surface. The sustained ability of Compound 3 to remain adhered to the cell membrane was evaluated by imaging the same cells as a function of time. It was found that Compound 3 remains confined to the cell membrane without labeling internal membranes of cells even up to 150 mins, outperforming the commercial standard CellMask™ which has a retention time of up to 90 mins in the plasma membrane (FIG. 3E). The positive charge on the trialkyl ammonium nitrogen on the acceptor group of the compound is likely responsible for its exceptionally stable and well-oriented localization on the cellular membrane. This orientation ensures that the hydrophobic donor side faces inward while the hydrophilic acceptor side extends outward uniformly across all dye molecules, without leading to cellular internalization. This behavior is similar to sulfofluoresceins in PeT dyes, where sulfonic acids facilitate proper orientation and prevent cellular internalization.

To test responsivity of Compound 3 to membrane potential, 500 nM Compound 3 labeled HeLa cells were imaged at different membrane potentials by holding the cell membrane at specific values of membrane potential using whole-cell patch clamp electrophysiology (FIGS. 3C-3D). Cells were held at −100 mV and imaged, and the procedure was repeated for increments of 40 mV up to +100 mV. The fluorescence intensity of Compound 3 decreased by ~9% per 100 mV as the membrane potential moved in the positive direction. This decrease is not due to photobleaching since control images of Compound 3 in resting HeLa cells showed negligible fluorescence changes. Such high photostability renders Compound 3 suitable for voltage imaging. While the voltage sensitivity of Compound 3 is comparable to that observed in other reported VSDs e.g., RH-421, ANNINE-6, and VF2.1.C1.,¹⁵ other asym-TTz dyes, its low toxicity, stable plasma membrane localization, photostability and ease of synthesis make it very attractive for future cellular applications.

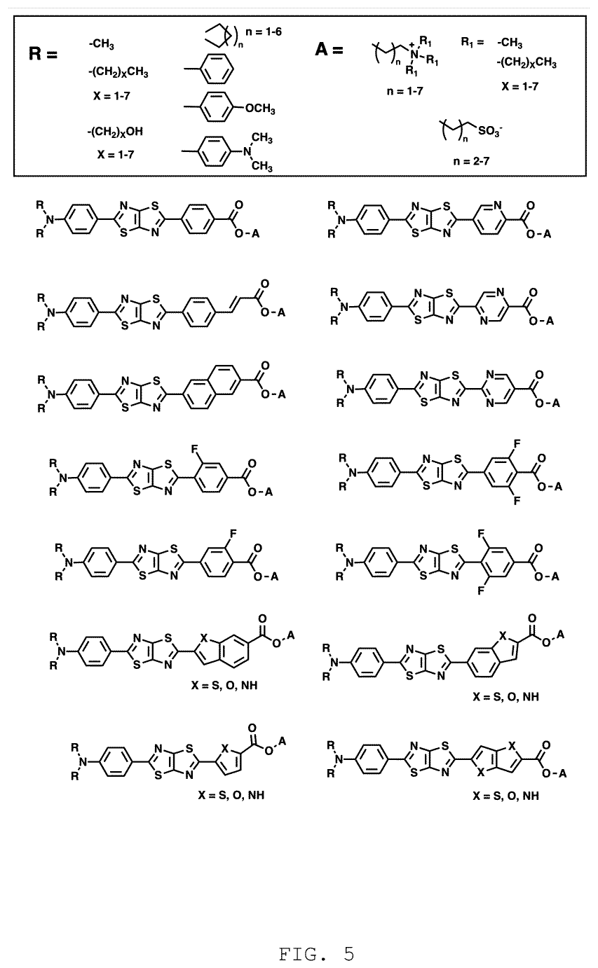

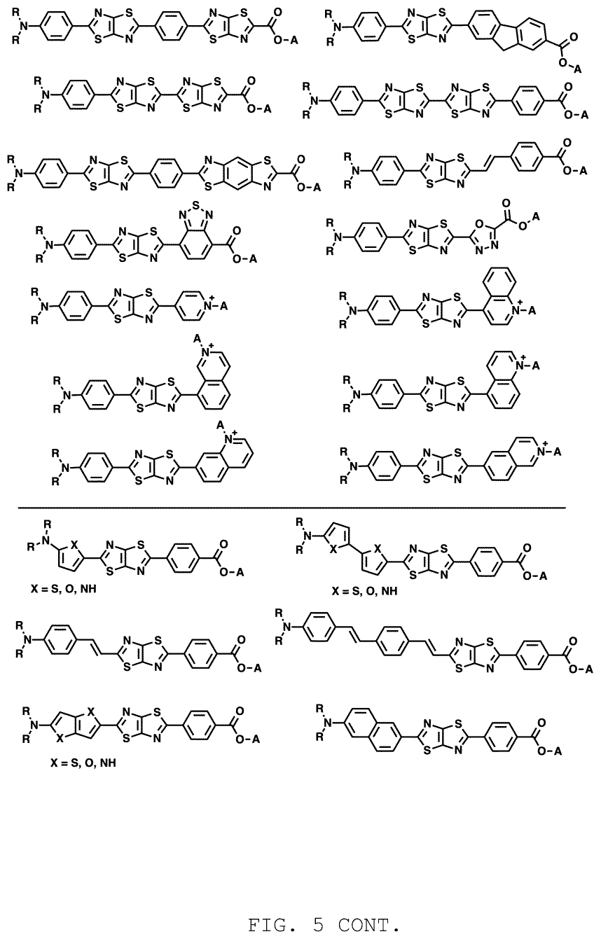

To understand the origin of the observed TTz dye voltage sensitivity, fluorescence emission spectra of Compound 3 labeled cells (FIG. 4A) was recorded at resting potential or at +100 mV. The latter was achieved by applying TEA-C1 (a K-channel blocker) and 200 mM NaCl.³⁴ Interestingly, Compound 3's fluorescence in cell membranes shows emission maxima at 487 nm, 517 nm, and a ~550 nm shoulder (FIG. 4A) that clearly reflects the vibronic structure of the dye. These vibronic structures are more well-resolved in non-polar solutions of asym-TTz dyes (FIG. 2C). It was found that the emission spectra changes depending on the membrane potential. At +100 mV, the 517 nm emission reduces. Notably, any spectral shifts in absorbance or emission was not observed that is generally observed with electrochromic dyes.

To explore how membrane potential affects fluorescence in this molecule, we performed time-dependent DFT (TD-DFT) calculations. While there were no appreciable changes in the molecular structure of the dye in different solvents, a significant change in the dihedral angles between the TTz core and the phenyl groups on the donor and the acceptor sides was observed in the excited states when we simulated applying an electric field along the long axis of the molecule (FIG. 4B). This mimics the electric field that the TTz dyes would experience within the cell membrane. Additionally, calculations show that the twist decreased the fluorescence intensity at higher values of membrane potential, and was more planar at lower values of membrane potential. FIG. 4B shows the difference in angles between the TTz core and the adjacent phenyl rings on the donor and acceptor sides in ground and excited states at +100 mV. Note that the twist is greater on the donor phenyl ring than on the acceptor phenyl ring, increasing from 0.5° in the ground state to 30.8° in the excited state on the donor side next to the donor sulfur of the TTz core. In contrast, the twist changes from 0.5° in the ground state to −7.9° in the excited state on the acceptor side next to the acceptor sulfur of the TTz core. In the twisted excited state, the light emission is less efficient since the p-orbitals are poorly aligned when twisted, thereby reducing π-conjugation. When the applied potential is more positive, it is computationally observed that the relative oscillator strength decreases, making the transition less favorable and thus affecting the fluorescence intensity. The reason why the peak at 517 nm in FIG. 4A decreases for the depolarized cell with respect to the one at resting potential is due to this peak's oscillator strength decreasing with increasing voltage, whilst the oscillator strength corresponding to the peak at 487 nm continues to remain steady with changing voltage. This fits the observed experimental data which shows that emission decreases as membrane potential becomes more positive and the twisted state is more favored.

Importantly, the foregoing characterizations reveal that the asym-TTz voltage sensing mechanism is unlike a PeT dye, where a fluorophore is quenched by an electron-rich molecular wire. It is unlike an electrochromic dye whereby the excitation and emission (ground state and the excited state) characteristics are shifted as membrane potential changes via the molecular Stark effect. The superior membrane localization and orientation of Compound 3 leads to membrane potential modulating its twist immediately on either side of the thiazolothiazole bridge in the excited state: a mechanism that modulates fluorescence emission intensity without shifting peak emission. This mechanism is likely enabled due to the unusually large difference between the ground state dipole moments (Δμt=6.45 D) and the excited state dipole moments (Δμt=30.2 D). The twisted excited state of the TTz-conjugated bridge enables this strong transition dipole to be leveraged for cell membrane voltage sensing. This unique system, with its innovative mechanism and ease of synthesis, not only enhances our understanding of the molecular behavior of TTz compounds—a relatively underexplored class of molecules—under varying membrane potentials, but also offers an unconventional way to observe cells due to its distinctive fluorescence properties. Most VSDs exhibit an increase in fluorescence during depolarization, and some electrochromic dyes show a decrease in fluorescence with higher membrane potentials. The ability to observe cells under hyperpolarization conditions with voltage sensitivity in this manner could pave the way for new research opportunities. Furthermore, this dye's voltage-sensing mechanism stands out as one of the simplest and most effective reported in the literature. It requires no molecular movement (unlike FRET), aggregation (unlike AIE), fluorescence changes dependent on elongated structures (unlike PeT), or hydrogen bonding (unlike ESIPT), and is highly sensitive (unlike electrochromism), making it arguably superior to other dyes in this field.

### Section 1: Materials and Methods

### Section 1.1: Materials and Instrumentation

4-(dibutylamino)benzaldehyde, dithiooxamide, methyl-4-formylbenzoate, (3-bromopropyl)trimethylammonium bromide, and all solvents used for spectroscopic measurements were purchased from Sigma-Aldrich and used without further purification. 1H-NMR measurements were obtained with either a JEOL 300 or 500 MHz NMR. High resolution mass spectra were obtained using a Voyager Matrix Assisted Laser Desorption/Ionization Time-of-Flight (MALDI-TOF) mass spectrometer, using 1,8,9-Trihydroxyanthracene as a matrix. Solution-state UV-Vis spectra were collected on a Cary 300 UV-Vis spectrophotometer. Time-resolved measurements were taken on a Horiba Scientific Fluoromax+ TCSPC spectrofluorometer equipped with a 405 nm diode laser for time-resolved PL decay measurements. All decays were calculated with a χ^{2~}1.2. Igor Pro 6.3 software was used to fit PL(t) decay data to single/multiple exponential decays. Quantum yields were calculated using perylene orange as a reference (quantum yield [Φ_{F}] in chloroform=0.99±0.05).
Cell culture. HeLa cells used in this investigation were generously provided by Dr. Nikolai Dulin (University of Chicago). These cells were cultured in Dulbecco's Modified Eagle's Medium (Invitrogen Corporation, USA) containing 10% heat inactivated Fetal Bovine Serum (FBS) (Invitrogen Corporation, USA), 100 U/mL penicillin, and 100 μg/mL streptomycin. Cells were maintained at 37° C. under 5% CO₂.
Section 1.1.1: Cell labeled spectral measurements. Excitation and emission spectra were collected by labeling the HeLa cells (0.5×10⁵) with 500 nM Bu₂N-TTz-CO₂ (NPr) in phosphate buffered saline (PBS) buffer and 0.1% TritonX-100. Following a 10-minute incubation period at 37° C., the cells were washed with PBS and transferred to a cuvette for fluorescent spectral analysis using a Fluoromax-4 spectrophotometer (Horiba Scientific, Edison, NJ, USA). Excitation spectra were recorded at an emission wavelength of 520 nm, while emission spectra were captured with an excitation wavelength of 440 nm.
Section 1.1.2: Electrophysiology. Whole-cell voltage-clamp measurements were conducted on HeLa cells labeled with 500 nM Bu₂N-TTz-CO₂ (NPr). The experiments utilized an Axopatch 200 A amplifier (Molecular Devices) for voltage clamping, with digitization performed using an NI-6251 DAQ (National Instruments), and control facilitated by WinWCP software (Strathclyde Electrophysiology Software). Imaging was carried out using an IX83 WF microscope at 40× magnification. Membrane seals were established using micropipettes filled with intracellular buffer, drawn from borosilicate glass capillaries pulled via Sutter P-97 Micropipette puller and polished with a Coater/Polisher Microforge (ALA Scientific Instruments Inc.). Patch pipettes with resistances between 5-8 MOhm were employed in the voltage-clamp experiments and positioned using an MP325 motorized manipulator (Sutter). Metamorph premier Ver. 7.8.12.0 was synchronized with an NI-6501 DAQ to enable voltage-triggered image acquisition. The extracellular solution composition was 145 mM NaCl, 10 mM HEPES, 3 mM KCl, 2 mM CaCl₂, 1 mM MgCl₂, 20 mM glucose, pH 7.4 (310 mOsm), while the intracellular solution composition was 115 mM potassium gluconate, 10 mM EGTA tetrapotassium salt, 10 mM HEPES, pH 7.2, 5 mM NaCl, 10 mM KCl, 2 mM ATP disodium salt (290 mOsm). After achieving whole-cell mode, the membrane was held at 0 mV, and step pulses were provided ranging from −100 mV to +100 mV with an increment of 40 mV at each step for 3.5 seconds. Simultaneously, at each voltage step, images were snapped for analysis.
Section 1.1.3: Microscopy. Imaging was carried out on an IX83 inverted microscope (Olympus Corporation of the Americas, Center Valley, PA, USA). This setup included a 100×, 1.4 NA, DIC oil immersion objective (PLAPON, Olympus) and an Evolve Delta 512 EMCCD camera (Photometrics, USA). Control over the Filter wheel, shutter, and CCD camera were facilitated by Metamorph premier Ver 7.8.12.0 (Molecular Devices, LLC, USA).
For photostability studies, HeLa cells were labeled with 500 nM Bu₂N-TTz-CO₂(NPr) for 10 mins followed by a wash with PBS. Subsequently, the cells were illuminated with a light source emitting ~5 W/cm² power. Images were captured at 1 min intervals using specific filter configurations: 430/24 bandpass excitation filter, 575/40 bandpass emission filter and Di02-R532 dichroic. VF2.1.C1 channel images were obtained using 525/30 bandpass excitation filter, 575/40 bandpass emission filter and 69002 dichroic. For CellMask™ red (Invitrogen Corporation, USA), images were obtained using the 640/30 bandpass excitation filter, 705/72 bandpass emission filter and 89007 dichroic.
Section 1.1.4: Cellular localization: To explore the prolonged membrane localization of Bu₂N-TTz-CO₂ (NPr), time-lapse imaging was employed. HeLa cells were initially treated with 500 nM Bu₂N-TTz-CO₂ (NPr) and a plasma membrane (CellMask™) for 10 minutes in 1×HBSS (Hank's balanced salt solution, Thermofisher). Subsequent to this labeling, any excess unbound dyes were eliminated via a wash with 1×PBS. The membrane-associated Bu₂N-TTz-CO₂ (NPr) and CellMask™ Red were sequentially imaged every 30 mins till 150 mins.
Section 1.1.5: Cytotoxicity: The cellular toxicity of the voltage-sensitive dye Bu₂N-TTz-CO₂ (NPr) was evaluated using a mitochondrial respiration assay involving dimethylthiazol diphenyl tetrazolium (MTT) staining (CyQuant MTT Cell Proliferation Assay Kit, Invitrogen). Initially, a 12 mM MTT stock solution was prepared by dissolving one 5 mg vial of MTT in 1 ml of sterile PBS, which was then vortexed or sonicated until fully dissolved. HeLa cells were cultured and treated with various concentrations of the dye, ranging from 0.05 to 500 μM, for 10 mins at 37° C., followed by a PBS wash.

Subsequently, 10 μl of the 12 mM MTT stock solution was added to 100 μl of freshly replaced culture media, followed by an incubation period at 37° C. for 2-5 h. After removing most of the medium from the wells, leaving only 25 μl, 50 μl of DMSO was added and thoroughly mixed by pipetting. Absorbance at 540 nm was measured after a further incubation with DMSO at 37° C. for 10 minutes. Based on these absorbance values, the normalized cell viability was calculated and plotted against the concentration of the dye used in the experiment.

### Section 1.2: Synthesis

Synthesis of asym-TTz molecules. Bu₂N-TTz-CO₂CH₃ was synthesized using a one-step mixed pot reaction and was hydrolyzed to form an acid and subsequently the Bu₂N-TTz-CO₂(NPr) derivative as is discussed in the manuscript.

### Section 1.2.1: 2-(N,N-dibutyl-4-aminophenyl)-5-(4-carbomethoxyphenyl) thiazolo[5,4-d]thiazole (Bu₂N-TTz-CO₂CH₃)

Dithiooxamide (0.175 g, 1.45 mmol), 4-(dibutylamino)benzaldehyde (0.373 g, 1.6 mmol), and 4-methyl-4-formylbenzaldehyde (0.263 g, 1.6 mmol) were refluxed in 10 mL of n-propanol (in air at 97° C.) for 48 h. The reaction was cooled to room temperature and left to sit in a fridge for 24 h. The product was collected using gravity filtration and rinsed with 30-40 mL ice-cold n-propanol to isolate an orange solid (0.151 g, 0.32 mmol, 22.07% yield). For molecular characterization (¹H-NMR, MALDI-TOF, and photophysical analyses), silica gel column chromatography using hexane:ethyl acetate, 6:1, was used to obtain a purified sample with negligible loss of crude product due to absence of side products. Only one fluorescent yellow band indicating the target product exclusively was observed during thin layer chromatography as well as silica gel column chromatography. This was further confirmed by the ¹H-NMR, MALDI-TOF, and thin layer chromatography results of the crude and the purified product being almost identical. ¹H NMR (300 MHz, d-CDCl₃, δ): 8.12 (d, J=8.52 Hz, 2H), 8.03 (d, J=8.52 Hz, 2H), 7.82 (d, J=8.79 Hz, 2H), 6.66 (d, J=9.06 Hz, 2H), 3.95 (s, 3H), 3.34 (t, J=7.70 Hz, 4H), 1.61 (m, 4H), 1.33 (m, 4H), 0.97 (t, J=7.29 Hz, 6H). UV-Vis λₘₐₓ (CHCl₃, M⁻¹ cm⁻¹): 430 nm (ε=45436). MALDI-TOF-MS: m/z calculated for C₂₆H₃₀N₃O₂S₂⁺ 480.18, found 480.72. Φ_{Hex}=0.98.

### Section 1.2.2: 2-(N,N-dibutyl-4-aminophenyl)-5-(4-caroboxyphenyl) thiazolo[5,4-d]thiazole (Bu₂N-TTz-CO₂H)

2-(N,N-dibutyl-4-aminophenyl)-5-(4-carbomethoxyphenyl) thiazolo[5,4-d]thiazole (1) (0.05 mmol, 0.025 g) was reacted with 2 M potassium hydroxide (5 mL), and 190 proof ethanol (5 mL, 88.54 mmol) for 96 h at 70° C. (reflux) in an aerated environment. The reaction was cooled to RT. The pH was then adjusted to ~2 using 1:1 conc. HCl:H₂O solution to obtain 0.02 g (0.04 mmol, 85.91% yield) of crude product. Using dichloromethane:methanol, 4:1, the crude product was purified by silica gel column chromatography. Only one fluorescent yellow band indicating the target product was observed. Thereafter, a yellow solid was obtained with negligible loss of crude product due to absence of side products. ¹H NMR (500 MHz, d-DMSO, δ): 8.00 (d, J=8.25 Hz, 2H), 7.92 (d, J=8.25 Hz, 2H), 7.73 (d, J=9.2 Hz, 2H), 6.68 (d, J=9.2 Hz, 2H), 3.29 (t, 4H), 1.46 (m, 2H), 1.28 (m, 2H), 0.86 (t, J=7.55 Hz, 6H). UV-Vis λₘₐₓ (CHCl₃, M⁻¹ cm⁻¹): 429 nm (ε=15215). MALDI-TOF-MS: m/z calculated for C₂₅H₂₈N₃O₂S₂⁺ 466.16, found 466.85. Φ_{CHCl3}=0.49.

### Section 1.2.3: 2-(N,N-dibutyl-4-aminophenyl)-5-(4-carbo[3-(trimethylammonio)propoxy]phenyl) thiazolo[5,4-d]thiazole bromide [Bu₂N-TTz-CO₂(NPr)]

2-(N,N-dibutyl-4-aminophenyl)-5-(4-carboxyphenyl) thiazolo[5,4-d]thiazole (2) (0.027 g, 0.06 mmol), (3-Bromopropyl)trimethylammonium bromide (0.094 g, 0.36 mmol) and potassium carbonate (0.066 mg, 0.48 mmol) were mixed in 30 mL acetone for 24 h at 52° C. The reaction mixture was cooled to RT and left overnight. Acetone was evaporated and the solid was rinsed with hexane and collected via gravity filtration (0.033 g, 0.06 mmol, 97.06% yield). Further purification was done by centrifuging the collected orange solid with hexanes, with negligible loss of crude product due to minimal formation of a fragment/side product that was very hard to observe even during molecular characterization studies. It could only be distinguished as a very weakly fluorescent spot under high intensity UV light during thin layer chromatography of the crude product. ¹H NMR (500 MHz, d-DCM, δ): 8.09 (d, J=8.7 Hz, 2H), 8.00 (d, J=8.7, 2H), 7.74 (d, J=9.15 Hz, 2H), 6.61 (d, J=9.15 Hz, 2H), 4.44 (t, J=5.95, 2H), 3.81 (t, J=8.48, 2H), 3.37 (s, 9H), 3.28 (m, 4H), 2.26 (m, 2H), 1.54 (m, 4H), 1.32 (m, 4H), 0.90 (t, J=7.33, 6H). UV-Vis λₘₐₓ (CHCl₃, M⁻¹ cm⁻¹): 437 nm. MALDI-TOF-MS: m/z calculated for C₃₁H₄₁N₄O₂S₂⁺ 565.27, found 565.88. Φ_{CHCl3}=0.40.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. An asymmetric thiazolothiazole-based compound of Formula I: 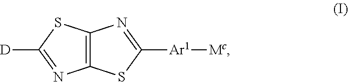
wherein D is an electron donor, Ar¹ is selected from the group consisting of arylene and heteroarylene, and M^{c} is a charged moiety.

2. The asymmetric thiazolothiazole-based compound of claim 1, wherein M^{c} is cationic.

3. The asymmetric thiazolothiazole-based compound of claim 2, wherein M^{c} comprises a quaternary ammonium group.

4. The asymmetric thiazolothiazole-based compound of claim 3, wherein M^{c} is of the formula: 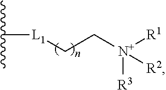
wherein L₁ is selected from the group consisting of a direct bond, —C(O)O—, —C(O)—, —R⁴—C(O)O—, and —R⁵—C(O)—, and wherein R¹-R³ are independently selected from the group consisting of hydrogen, alkyl, cycloalkyl, and alkenyl, and wherein R⁴ and R⁵ are independently selected from the group consisting of alkyl and alkenyl, and wherein n is an integer from 0 to 10.

5. The asymmetric thiazolothiazole-based compound of claim 1, wherein M^{c} is anionic.

6. The asymmetric thiazolothiazole-based compound of claim 4, wherein M^{c} comprises an anionic acid group.

7. The asymmetric thiazolothiazole-based compound of claim 5, wherein the anionic acid group is selected from the group consisting of carboxylate and sulfonate.

8. The asymmetric thiazolothiazole-based compound of claim 6, wherein M^{c} is of the formula: 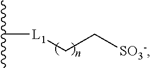
wherein L₁ is selected from the group consisting of a direct bond, —C(O)O—, —C(O)—, —R¹—C(O)O—, and —R²—C(O)—, and wherein R¹ and R² are independently selected from the group consisting of alkyl and alkenyl, and wherein n is an integer from 0 to 10.

9. The asymmetric thiazolothiazole-based compound of claim 1, wherein D is hydrophobic.

10. The asymmetric thiazolothiazole-based compound of claim 1, wherein D is selected from the group consisting of aryl and heteroaryl.

11. The asymmetric thiazolothiazole-based compound of claim 9, wherein the aryl and heteroaryl comprise one or more substituents selected from the group consisting of alkylamine and arylamine.

12. The asymmetric thiazolothiazole-based compound of claim 1, wherein D is of the formula: 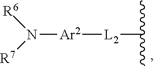
wherein R⁶ and R⁷ are independently selected from the group consisting of hydrogen, alkyl, cycloalkyl, alkenyl, alcohol, aryl, and heteroaryl.

13. The asymmetric thiazolothiazole-based compound of claim 11, wherein D is of the formula: 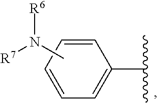
wherein R⁶ and R⁷ are independently selected from the group consisting of hydrogen, alkyl, cycloalkyl, alkenyl, alcohol, aryl, and heteroaryl.

14. The asymmetric thiazolothiazole-based compound of claim 1 exhibiting a change in dipole moment between ground and excited states of at least 20 D.

15. The asymmetric thiazolothiazole-based compound of claim 1 having a peak absorption and peak emission in the visible region of the electromagnetic spectrum.

16. The asymmetric thiazolothiazole-based compound of claim 14, wherein the peak absorption is in a range of 400 nm to 500 nm.

17. The asymmetric thiazolothiazole-based compound of claim 1 exhibiting solvatofluorochromism.

18. The asymmetric thiazolothiazole-based compound of claim 1, wherein fluorescence of the asymmetric thiazolothiazole-based compound increases or decreases relative to voltage changes in a local environment of the asymmetric thiazolothiazole-based compound.

19. The asymmetric thiazolothiazole-based compound of claim 1 having a difference between the highest occupied molecular orbital (HOMO) and lowest unoccupied molecular orbital (LUMO) of at least 1 eV.

20. The asymmetric thiazolothiazole-based compound of claim 18, wherein the difference is 1 eV to 3 eV.

21. The asymmetric thiazolothiazole-based compound of claim 1 exhibiting a Stokes shift of at least 0.3 eV.

22. A method of making a thiazolothiazole-based compound comprising:
reacting dithiooxamide with a first aryl aldehyde and a second aryl aldehyde in a protic polar organic solvent to provide the thiazolothiazole-based compound; and
isolating the thiazolothiazole-based compound without chromatographic separation.

23. The method of claim 21, wherein the first and second aryl aldehydes have differing structures.

24. The method of claim 21, wherein the protic polar organic solvent has a boiling point less than 100° C.

25. The method of claim 21, wherein the protic polar organic solvent is an alcohol.

26. The method of claim 21, wherein the reaction time is at least 24 hours.

27. The method of claim 21 wherein the dithiooxamide, first aryl aldehyde and second aryl aldehyde are refluxed in the protic polar organic solvent.

28. The method of claim 21 further comprising functionalizing the thiazolothiazole-based compound with a cationic or anionic moiety.

29. The method of claim 27, wherein the cationic moiety comprises a quaternary ammonium group.

30. The method of claim 27, wherein the anionic moiety comprises an acid group selected from the group consisting of carboxylate and sulfonate.

31. The method of claim 21, wherein the first aryl aldehyde is an electron donor precursor, and the second aryl aldehyde is an electron acceptor precursor.

32. A biological composition comprising:
cells or cellular components; and
a marker associated with membranes of the cells or cellular components, the marker comprising a thiazolothiazole-based compound of Formula I: 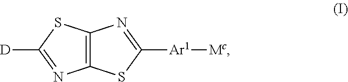
wherein D is an electron donor, Ar¹ is selected from the group consisting of arylene and heteroarylene, and M^{c} is a charged moiety.

33. The biological composition of claim 30, wherein the thiazolothiazole-based compound inserts into a phospholipid membrane of the cells or cellular composition.

34. The biological composition of claim 31, wherein M^{c} interacts with one or more charged moieties of the phospholipid membrane surface.

35. The biological composition of claim 33, wherein the one or more charged moieties comprise phosphate groups or quaternary ammonium groups.

36. The biological composition of claim 31, wherein M^{c} is of the formula: 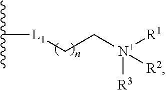
wherein L₁ is selected from the group consisting of a direct bond, —C(O)O—, —C(O)—, —R⁴—C(O)O—, and —R⁵—C(O)—, and wherein R¹-R³ are independently selected from the group consisting of hydrogen, alkyl, cycloalkyl, and alkenyl, and wherein R⁴ and R⁵ are independently selected from the group consisting of alkyl and alkenyl, and wherein n is an integer from 0 to 10.

37. The biological composition of claim 31, wherein M^{c} comprises an anionic acid group.

38. The biological composition of claim 36, wherein the anionic acid group is selected from the group consisting of carboxylate and sulfonate.

39. The biological composition of claim 36, wherein M^{c} is of the formula: 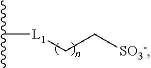
wherein L₁ is selected from the group consisting of a direct bond, —C(O)O—, —C(O)—, —R¹—C(O)O—, and —R²—C(O)—, and wherein R¹ and R² are independently selected from the group consisting of alkyl and alkenyl, and wherein n is an integer from 0 to 10.

40. The biological composition of claim 31, wherein D is hydrophobic.

41. The biological composition of claim 31, wherein D is selected from the group consisting of aryl and heteroaryl.

42. The biological composition of claim 40, wherein the aryl and heteroaryl comprise one or more substituents selected from the group consisting of alkylamine and arylamine.

43. The biological composition of claim 40, wherein D is of the formula: 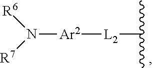
wherein R⁶ and R⁷ are independently selected from the group consisting of hydrogen, alkyl, cycloalkyl, alkenyl, alcohol, aryl, and heteroaryl.

44. The biological composition of claim 42, wherein D is of the formula: 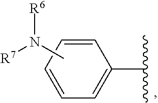
wherein R⁶ and R⁷ are independently selected from the group consisting of hydrogen, alkyl, cycloalkyl, alkenyl, alcohol, aryl, and heteroaryl.

45. The biological composition of claim 31 exhibiting a change in dipole moment between ground and excited states of at least 20 D.

46. The biological composition of claim 31 having a peak absorption and peak emission in the visible region of the electromagnetic spectrum.
